(19) United States
(12) Patent Application Publication
(10) Pub. No.: US 2016/0174942 A1
(43) Pub. Date: Jun. 23, 2016

(54) METHOD AND SYSTEM FOR ENHANCED VISUALIZATION BY AUTOMATICALLY ADJUSTING ULTRASOUND IMAGE COLOR AND CONTRAST

(71) Applicant: General Electric Company, Schenectady, NY (US)

(72) Inventors: Per Arne Linnerud, Horten (NO); Erik Normann Steen, Moss (NO)

(21) Appl. No.: 14/580,575

(22) Filed: Dec. 23, 2014

(51) Int. Cl. A61B 8/08; A61B 8/00; A61B 8/13

(52) U.S. Cl. CPC A61B 8/5207; A61B 8/13; A61B 8/461; A61B 8/4444

(57) ABSTRACT

Certain embodiments provide a method and system for enhancing visualization of an image acquired with a handheld ultrasound scanner by automatically adjusting the image to optimize the color and contrast. The method may include performing, by a probe of an ultrasound system, an ultrasound scan to acquire ultrasound scan data. The method may also include detecting, by a sensor, lighting conditions. The method may include measuring, by a processor of the ultrasound system, a characteristic of the detected lighting conditions. The method may also include selecting, by the processor, a color map based on the measured characteristic. The method may include applying, by the processor, the selected color map to the acquired ultrasound scan data to generate processed ultrasound scan data.

### CROSS-REFERENCE TO RELATED APPLICATIONS/INCORPORATION BY REFERENCE

[Not Applicable]

### FIELD OF THE INVENTION

Certain embodiments of the invention relate to ultrasound imaging. More specifically, certain embodiments of the invention relate to a method and system for enhanced visualization of an image acquired with a handheld ultrasound scanner by automatically adjusting the image to optimize the color and contrast.

### BACKGROUND OF THE INVENTION

Ultrasound imaging is a medical imaging technique for imaging organs and soft tissues in a human body. Ultrasound imaging uses real time, non-invasive high frequency sound waves to produce a two-dimensional (2D) image and/or a three-dimensional (3D) image.

Handheld ultrasound scanners are commonly used to acquire ultrasound data in emergency situations, such as in ambulances, helicopters, and other outdoor environments with fast changing and/or difficult light conditions. For example, helicopters may use red lighting to preserve the night vision of the pilots. As another example, an ambulance may drive in and out of tunnels, causing drastic changes to lighting conditions in a fraction of a second. In emergency situations, an operator of the handheld ultrasound scanner may not have time to manually change display parameters or color maps to adapt to the changing conditions. Further, the operators of handheld ultrasound scanners in emergency situations may have less training in sonography than specialists at a hospital, for example. Consequently, less trained users may have difficulty navigating menus to select an optimal color map and may not know which of the available color maps is best for a particular lighting condition.

Existing display devices, such as mobile phones, have attempted to improve image visualization by providing automatic backlight adjustment. While backlight adjustment may improve visualization in a limited light range, a strong backlight does not provide good contrast in strong outdoor light. The use of a strong backlight also consumes a high amount of power, which is problematic for the limited battery capacity of handheld ultrasound scanners.

Further limitations and disadvantages of conventional and traditional approaches will become apparent to one of skill in the art, through comparison of such systems with some aspects of the present invention as set forth in the remainder of the present application with reference to the drawings.

### BRIEF SUMMARY OF THE INVENTION

A system and/or method is provided for enhanced visualization of an image acquired with a handheld ultrasound scanner by automatically adjusting the image to optimize the color and contrast, substantially as shown in and/or described in connection with at least one of the figures, as set forth more completely in the claims.

These and other advantages, aspects and novel features of the present invention, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to provide enhanced visualization of an image acquired with a handheld ultrasound scanner by automatically adjusting the image to optimize the color and contrast, in accordance with an embodiment of the invention.

FIG. 2 is a flow chart illustrating exemplary steps that may be utilized for providing enhanced visualization of an image acquired with a handheld ultrasound scanner by automatically adjusting the image to optimize the color and contrast, in accordance with an embodiment of the invention.

### DETAILED DESCRIPTION OF THE INVENTION

Certain embodiments of the invention may be found in a method and system for adjusting the color and contrast of an image acquired by a handheld ultrasound scanner. For example, aspects of the present invention have the technical effect of quickly and automatically selecting a color map based on detected light conditions and applying the color map to acquired ultrasound image data, thereby improving the workflow of the operator while enhancing visualization of the image data.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments of the present invention. The following detailed description is, therefore, not to be taken in a limiting sense, and the scope of the present invention is defined by the appended claims and their equivalents.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode, CF-mode and/or sub-modes of CF such as TVI, Angio, B-flow, BMI, BMI_Angio, and in some cases also MM, CM, PW, TVD, CW where the “image” and/or “plane” includes a single beam or multiple beams.

Furthermore, the term processor or processing unit, as used herein, refers to any type of processing unit that can carry out the required calculations needed for the invention, such as single or multi-core: CPU, Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

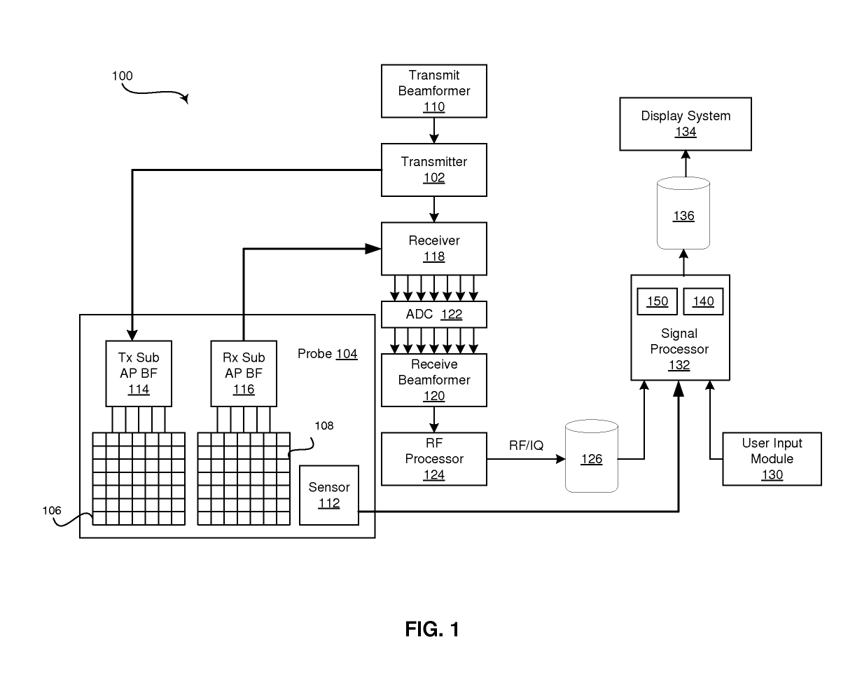

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to provide enhanced visualization of an image acquired with a handheld ultrasound scanner by automatically adjusting the image to optimize the color and contrast, in accordance with an embodiment of the invention. Referring to FIG. 1, there is shown an ultrasound system 100. The ultrasound system 100 comprises a transmitter 102, an ultrasound probe 104, a transmit beamformer 110, a receiver 118, a receive beamformer 120, a RF processor 124, a RF/IQ buffer 126, a user input module 130, a signal processor 132, an image buffer 136, and a display system 134.

The transmitter 102 may comprise suitable logic, circuitry, interfaces and/or code that may be operable to drive an ultrasound probe 104. The ultrasound probe 104 may comprise a one dimensional (1D, 1.25D, 1.5D or 1.75D) array or a two dimensional (2D) array of piezoelectric elements. The ultrasound probe 104 may comprise a group of transmit transducer elements 106 and a group of receive transducer elements 108, that normally constitute the same elements. The ultrasound probe 104 may comprise a sensor 112 for determining current lighting conditions. Additionally or alternatively, the sensor 112 may be separately housed or disposed with other components of the system, such as the signal processor 132. The sensor 112 may be, for example, a red green blue (RGB) light color sensor that provides information regarding the amount of color in detected light, or any suitable sensor. The sensor 112 may provide the detected light information to the light measurement module 150 of the signal processor 132 for color measurement and/or any suitable processing to determine the current lighting conditions.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells, and surgical instruments in the object of interest to produce echoes. The echoes are received by the receive transducer elements 108. The group of receive transducer elements 108 in the ultrasound probe 104 may be operable to convert the received echoes into analog signals, undergo sub-aperture beamforming by a receive sub-aperture beamformer 116 and are then communicated to a receiver 118.

The receiver 118 may comprise suitable logic, circuitry, interfaces and/or code that may be operable to receive and demodulate the signals from the receive sub-aperture beamformer 116. The demodulated analog signals may be communicated to one or more of the plurality of A/D converters 122. The plurality of A/D converters 122 may comprise suitable logic, circuitry, interfaces and/or code that may be operable to convert the demodulated analog signals from the receiver 118 to corresponding digital signals. The plurality of A/D converters 122 are disposed between the receiver 118 and the receive beamformer 120. Notwithstanding, the invention is not limited in this regard. Accordingly, in some embodiments of the invention, the plurality of A/D converters 122 may be integrated within the receiver 118.

The receive beamformer 120 may comprise suitable logic, circuitry, interfaces and/or code that may be operable to perform digital beamforming processing on the signals received from the plurality of A/D converters 122. The resulting processed information may be converted back to corresponding RF signals. The corresponding output RF signals that are output from the receive beamformer 120 may be communicated to the RF processor 124. In accordance with some embodiments of the invention, the receiver 118, the plurality of A/D converters 122, and the beamformer 120 may be integrated into a single beamformer, which may be digital.

The RF processor 124 may comprise suitable logic, circuitry, interfaces and/or code that may be operable to demodulate the RF signals. In accordance with an embodiment of the invention, the RF processor 124 may comprise a complex demodulator (not shown) that is operable to demodulate the RF signals to form I/Q data pairs that are representative of the corresponding echo signals. The RF or I/Q signal data may then be communicated to an RF/IQ buffer 126.

The RF/IQ buffer 126 may comprise suitable logic, circuitry, interfaces and/or code that may be operable to provide temporary storage of the RF or I/Q signal data, which is generated by the RF processor 124.

The user input module 130 may be utilized to input patient data, surgical instrument data, scan parameters, settings, configuration parameters, change scan mode, and the like. In an exemplary embodiment of the invention, the user input module 130 may be operable to configure, manage and/or control operation of one or more components and/or modules in the ultrasound system 100. In this regard, the user input module 130 may be operable to configure, manage and/or control operation of transmitter 102, the ultrasound probe 104, the transmit beamformer 110, the receiver 118, the receive beamformer 120, the RF processor 124, the RF/IQ buffer 126, the user input module 130, the signal processor 132, the image buffer 136, and/or the display system 134

The signal processor 132 may comprise suitable logic, circuitry, interfaces and/or code that may be operable to process ultrasound scan data (i.e., RF signal data or IQ data pairs) for generating an ultrasound image for presentation on a display system 134. The signal processor 132 is operable to perform one or more processing operations according to a plurality of selectable ultrasound modalities on the acquired ultrasound scan data. In an exemplary embodiment of the invention, the signal processor 132 may be operable to perform compounding, motion tracking, and/or speckle tracking. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation.

In various embodiments, the signal processor may comprise a light measurement module 150 and a color map processing module 140. The light measurement module 150 may comprise suitable logic, circuitry, interfaces and/or code that may be operable to handle processing data from the sensor 112. For example, the light measurement module 150 may be operable to measure the color present in the current lighting conditions detected by the sensor 112. The light measurement module 150, or color map processing module 140, can select a color map from a plurality of available color maps based on the measurement to provide optimal contrast. In various embodiments, the color map selection may be determined based on an algorithm applying one or more color theory principles, such as complementary color schemes, eye color sensitivity, and color sensitivity as a function of the general light intensity (i.e., the Purkinje effect), among other things. For example, a strong outdoor light often contains large quantities of scattered blue light. Accordingly, a color map corresponding with the complimentary color orange, which is on the opposite side of the color wheel from blue, may be selected for application to the acquired ultrasound image data to provide high contrast in the subsequently displayed image data. As another example, a sensitivity shift from strong outdoor light to lower light levels may cause a transition into yellow, green, and then blue color maps, based on the Purkinje effect color theory principle. Further, the eye is generally more sensitive to yellow and green color maps, which may factor into the color map selection. The color map processing module 140 may comprise suitable logic, circuitry, interfaces and/or code that may be operable to process the acquired ultrasound data to apply the selected color map.

The ultrasound system 100 may be operable to continuously acquire ultrasound scan data at a frame rate that is suitable for the imaging situation in question. Typical frame rates range from 20-70 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several seconds worth of frames of ultrasound scan data. The frames of ultrasound scan data are stored in a manner to facilitate retrieval thereof according to its order or time of acquisition. The image buffer 136 may be embodied as any known data storage medium.

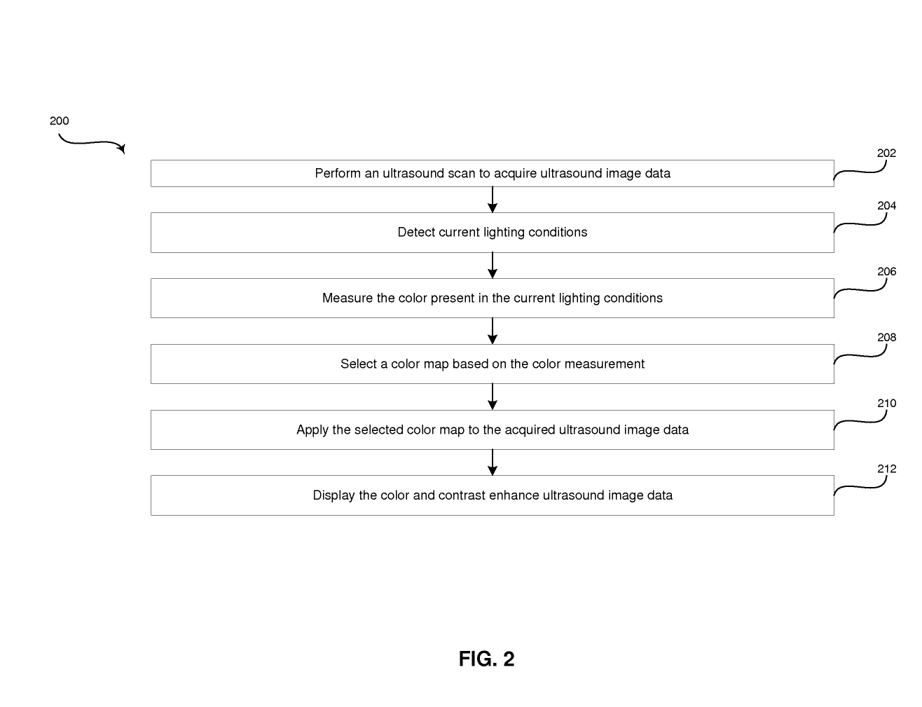

FIG. 2 is a flow chart illustrating exemplary steps that may be utilized for providing enhanced visualization of an image acquired with a handheld ultrasound scanner 100 by automatically adjusting the image to optimize the color and contrast, in accordance with an embodiment of the invention. Referring to FIG. 2, there is shown a flow chart 200 comprising exemplary steps 202 through 212. Certain embodiments of the present invention may omit one or more of the steps, and/or perform the steps in a different order than the order listed, and/or combine certain of the steps discussed below. For example, some steps may not be performed in certain embodiments of the present invention. As a further example, certain steps may be performed in a different temporal order, including simultaneously, than listed below.

In step 202, an ultrasound scan is performed to acquire ultrasound image data. For example, the ultrasound probe 104 in the ultrasound system 100 may be operable to perform an ultrasound scan of patient anatomy. The ultrasound image data may be grayscale B-mode or M-mode image data, or any suitable ultrasound image data.

In step 204, current lighting conditions may be detected. For example, a sensor 112 may receive information regarding the lighting conditions in the vicinity of the ultrasound scan. The sensor 112 may be disposed in or on the probe 104, housed separately and connected to ultrasound system 100 as a user input module 130, and/or disposed with other components of the ultrasound system 100, such as the signal processor 132, among other things. The sensor 112 may be an RGB light color sensor that is operable to detect the amount of color in the current lighting conditions, or any suitable sensor configured to detect current lighting conditions. The detected lighting information may be communicated to a signal processor 132.

In step 206, a light measurement module 150 of the signal processor 132 of the ultrasound system 100 may perform a measurement of the lighting information received from the sensor 112. For example, the light measurement module 150 may measure the color present in the current lighting conditions. In various embodiments, the measurement may correspond with a color on a color wheel representing relationships between colors based on well-known color theory principles.

In step 208, the light measurement module 150 or a color map processing module 140 of the signal processor 132, may select a color map based on the measurement performed at step 206. For example, a plurality of color maps may be available for selection based on the current lighting conditions. The signal processor 132 can select a color map from a plurality of available color maps based on the measurement to provide optimal contrast. The color map selection may be determined based on, for example, an algorithm applying a complementary color scheme to the measured color, eye color sensitivity, color sensitivity as a function of the general light intensity (i.e., the Purkinje effect), and/or any suitable color theory principle. For example, if the sensor 112 provides information corresponding to a strong outdoor light and the light measurement module 150 measures large quantities of scattered blue light, a color map corresponding with the complimentary color orange, which is on the opposite side of the color wheel from blue, may be selected by the light measurement module 150 or a color map processing module 140. As another, example, if the light measurement module 150 measures lower light levels detected by the sensor 112 in an interior of an emergency helicopter, the signal processor 132 may select a yellow, green, or blue color map, based on the Purkinje effect color theory principle. Further, the eye is generally more sensitive to yellow and green color maps, which may factor into the color map selection by the signal processor 132.

In step 210, a color map processing module 140 of a signal processor 132 of the ultrasound system 100 can apply the color map selected at step 208 to the ultrasound image data acquired at step 202. For example, if grayscale B-mode image data was acquired at step 202 and an orange color map was selected at step 208 after the signal processor 132 measured large quantities of scattered blue light at step 206 detected by sensor 112 at step 204, the orange color map is applied by the color map processing module 140 to the grayscale B-mode image data at step 210 to provide a processed ultrasound image with optimal color and contrast for the current viewing conditions. In step 212, the color and contrast enhanced ultrasound image data may be displayed at display system 134, or the like.

Aspects of the present invention provide a method 200 and system 100 for enhancing the visualization of an image acquired with a handheld ultrasound scanner by automatically adjusting the image to optimize the color and contrast. In accordance with various embodiments of the invention, the method 200 comprises performing 202, by a probe 104 of an ultrasound system 100, an ultrasound scan to acquire ultrasound scan data. The method 200 comprises detecting 204, by a sensor 112, lighting conditions. The method 200 comprises measuring 206, by a processor 132, 150 of the ultrasound system 100, a characteristic of the detected lighting conditions. The method 200 comprises selecting 208, by the processor 132, 140, 150, a color map based on the measured characteristic. The method 200 comprises applying 210, by the processor 132, 140, the selected color map to the acquired ultrasound scan data to generate processed ultrasound scan data.

In a representative embodiment, the method 200 comprises presenting 212, at a display system 134, the processed ultrasound scan data. In various embodiments, the acquired ultrasound scan data is one of a grayscale B-mode image and a grayscale M-mode image. In certain embodiments, the sensor 112 is a red, green, blue (RGB) light color sensor. In a representative embodiment, the characteristic is one or more of color and intensity. In various embodiments, the selected color map corresponds with a color that is one or more of complimentary to the measured color, a function of the intensity, and sensitive to human vision. In certain embodiments, the sensor 112 is disposed one or more of in or on the probe 104.

Various embodiments provide a system comprising an ultrasound device 100. The ultrasound device 100 comprises a probe 104 configured to acquire ultrasound scan data. The ultrasound device 100 comprises a sensor 112 configured to detect lighting conditions. The ultrasound device 100 comprises a processor 132, 150 configured to measure a characteristic of the detected lighting conditions. The processor 132, 140, 150 is configured to select a color map based on the measured characteristic. The processor 132, 140 is configured to apply the selected color map to the acquired ultrasound scan data to generate processed ultrasound scan data.

In certain embodiments, the ultrasound device 100 comprises a display system 134 configured to present the processed ultrasound scan data. In a representative embodiment, the acquired ultrasound scan data is one of a grayscale B-mode image and a grayscale M-mode image. In various embodiments, the sensor 112 is a red, green, blue (RGB) light color sensor. In certain embodiments, the characteristic is one or more of color and intensity. In a representative embodiment, the selected color map corresponds with a color that is one or more of complimentary to the measured color, a function of the intensity, and sensitive to human vision. In various embodiments, the sensor 112 is disposed one or more of in or on the probe 104. In certain embodiments, the processor 132, 140, 150 is configured to select the color map from a plurality of color maps. In a representative embodiment, each of the plurality of color maps corresponds with a different one of a plurality of lighting conditions.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled, or not enabled, by some user-configurable setting.

Other embodiments of the invention may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for enhanced visualization of an image acquired with a handheld ultrasound scanner by automatically adjusting the image to optimize the color and contrast.

Accordingly, the present invention may be realized in hardware, software, or a combination of hardware and software. The present invention may be realized in a centralized fashion in at least one computer system, or in a distributed fashion where different elements are spread across several interconnected computer systems. Any kind of computer system or other apparatus adapted for carrying out the methods described herein is suited. A typical combination of hardware and software may be a general-purpose computer system with a computer program that, when being loaded and executed, controls the computer system such that it carries out the methods described herein.

The present invention may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

What is claimed is:

1. A method, comprising:
performing, by a probe of an ultrasound system, an ultrasound scan to acquire ultrasound scan data;
detecting, by a sensor, lighting conditions;
measuring, by a processor of the ultrasound system, a characteristic of the detected lighting conditions;
selecting, by the processor, a color map based on the measured characteristic; and
applying, by the processor, the selected color map to the acquired ultrasound scan data to generate processed ultrasound scan data.

2. The method according to claim 1, comprising presenting, at a display system, the processed ultrasound scan data.

3. The method according to claim 1, wherein the acquired ultrasound scan data is one of:
a grayscale B-mode image, and
a grayscale M-mode image.

4. The method according to claim 1, wherein the sensor is a red, green, blue (RGB) light color sensor.

5. The method according to claim 1, wherein the characteristic is one or more of color and intensity.

6. The method according to claim 5, wherein the selected color map corresponds with a color that is one or more of:
complimentary to the measured color,
a function of the intensity, and
sensitive to human vision.

7. The method according to claim 1, wherein the sensor is disposed one or more of in or on the probe.

8. A system, comprising:
an ultrasound device comprising:
a probe configured to acquire ultrasound scan data;
a sensor configured to detect lighting conditions; and
a processor configured to:
measure a characteristic of the detected lighting conditions,
select a color map based on the measured characteristic, and
apply the selected color map to the acquired ultrasound scan data to generate processed ultrasound scan data.

9. The system according to claim 8, wherein the ultrasound device comprises a display system configured to present the processed ultrasound scan data.

10. The system according to claim 8, wherein the acquired ultrasound scan data is one of:
a grayscale B-mode image, and
a grayscale M-mode image.

11. The system according to claim 8, wherein the sensor is a red, green, blue (RGB) light color sensor.

12. The system according to claim 8, wherein the characteristic is one or more of color and intensity.

13. The system according to claim 12, wherein the selected color map corresponds with a color that is one or more of:
complimentary to the measured color,
a function of the intensity, and
sensitive to human vision.

14. The system according to claim 8, wherein the sensor is disposed one or more of in or on the probe.

15. The system according to claim 8, wherein the processor is configured to select the color map from a plurality of color maps, each of the plurality of color maps corresponding with a different one of a plurality of lighting conditions.

16. A non-transitory computer readable medium having stored thereon, a computer program having at least one code section, the at least one code section being executable by a machine for causing the machine to perform steps comprising:
receiving acquired ultrasound scan data;
receiving detected lighting conditions;
measuring a characteristic of the detected lighting conditions;
selecting a color map based on the measured characteristic; and
applying the selected color map to the acquired ultrasound scan data to generate processed ultrasound scan data.

17. The non-transitory computer readable medium according to claim 16, comprising presenting the processed ultrasound scan data.

18. The non-transitory computer readable medium according to claim 16, wherein the acquired ultrasound scan data is one of:
a grayscale B-mode image, and
a grayscale M-mode image.

19. The non-transitory computer readable medium according to claim 15, wherein the characteristic is one or more of color and intensity.

20. The non-transitory computer readable medium according to claim 19, wherein the selected color map corresponds with a color that is one or more of:
complimentary to the measured color,
a function of the intensity, and
sensitive to human vision.
